(19) United States
(12) Patent Application Publication
(10) Pub. No.: US 2004/0044080 A1
(43) Pub. Date: Mar. 4, 2004

(54) Treatment of dyspareunia with topically administered nitroglycerin formulations

(76) Inventors: Virgil A. Place, Kawaihae, HI (US); Leland F. Wilson, Menlo Park, CA (US); Paul C. Doherty JR., Cupertino, CA (US); Mark S. Hanamoto, Belmont, CA (US); Alfred P. Spivack, Menlo Park, CA (US); Neil Gesundheit, Los Altos, CA (US); Sean R. Bennett, Denver, CO (US); Jane K. Doherty, Cupertino, CA (US)

Correspondence Address: REED & EBERLE LLP, 800 MENLO AVENUE, SUITE 210, MENLO PARK, CA, 94025, US

(21) Appl. No.: 10/407,858

(22) Filed: Apr. 4, 2003

Related U.S. Application Data
(63) Continuation-in-part of application No. 09/905,458, filed on Jul. 13, 2001, now Pat. No. 6,593,313.
Application No. 09/905,458 is a continuation of application No. 09/539,484, filed on Mar. 30, 2000, now Pat. No. 6,306,841.
Application No. 09/539,484 is a continuation of application No. 09/181,316, filed on Oct. 27, 1998, now abandoned.
Application No. 09/181,316 is a continuation-in-part of application No. 08/959,064, filed on Oct. 28, 1997, now Pat. No. 5,877,216.
Application No. 09/181,316 is a continuation-in-part of application No. 08/959,057, filed on Oct. 28, 1997, now abandoned.

(51) Int. Cl. A61K 31/21; A61K 31/557

(52) U.S. Cl. 514/573; 514/509

(57) ABSTRACT

Methods and formulations for treating dyspareunia are provided. A pharmaceutical composition formulated so as to contain a therapeutically effective amount of nitroglycerin is administered to the vagina or vulvar area of the individual undergoing treatment. Preferred formulations are immediate release formulations in which at least 80% of the nitroglycerin in the formulation is released therefrom within 4 hours following administration. The formulations may contain one or more additional active agents, e.g., agents that are also useful to treat dyspareunia and/or potentiate the action of nitroglycerin. Such additional agents include vasoactive agents such as prostaglandins, phosphodiesterase inhibitors, androgens such as testosterone, estrogens such as estradiol, and selective modulators of estrogen and androgen receptors. A kit for a patient to use in the self-administration of the formulation is also provided.

### CROSS-REFERENCE TO RELATED APPLICATIONS

This application is a continuation-in-part of U.S. Ser. No. 09/905,458, filed Jul. 13, 2001; which was a continuation of U.S. Ser. No. 09/539,484, filed Mar. 30, 2000, now U.S. Pat. No. 6,306,841; which was a continuation of U.S. Ser. No. 09/181,316, filed Oct. 27, 1998, now abandoned; which was a continuation-in-part of both U.S. Ser. No. 08/959,064, filed Oct. 28, 1997, now U.S. Pat. No. 5,877,216, and U.S. Ser. No. 08/959,057, filed Oct. 28, 1997, now abandoned; the disclosures of which are hereby incorporated by reference.

### TECHNICAL FIELD

This invention relates generally to methods and pharmaceutical formulations for treating women suffering from dyspareunia. More particularly, the invention pertains to the topical administration of a nitroglycerin-containing pharmaceutical formulation in the treatment of dyspareunia.

### BACKGROUND

Sexual response in women is generally classified into four stages: excitement, plateau, orgasm, and resolution. Masters and Johnson, Human Sexual Response (Boston, Mass.: Little, Brown & Co., 1966). With sexual arousal and excitement, vasocongestion and muscular tension increase progressively, primarily in the genitals, and is manifested by increased blood flow, elevated luminal oxygen tension, and vaginal surface lubrication as a result of plasma transudation that saturates the fluid reabsorptive capacity of the vaginal epithelium. Vasoactive intestinal polypeptide (“VIP”) release may induce the physiological changes of sexual arousal and excitement, and may be the major neurotransmitter that participates in the innervation of the vaginal blood supply. Peptide histidine methionine has been co-located with VIP within nerve fibers that innervate small blood vessels, smooth muscle and epithelial cells in the vaginal tract.

Sexual excitement can be initiated by numerous psychogenic or somatogenic stimuli and must be reinforced to result in orgasm. With continued stimulation, excitement progresses in intensity into a plateau stage, from which the individual can shift into orgasm. The orgasmic stage is characterized by a rapid release from vasocongestion and muscular tension.

During the various stages of sexual response, characteristic genital and extragenital responses occur. Estrogens magnify the sexual responses; however, sexual responses may also occur in estrogen-deficient individuals. Sexual dysfunction may be due to organic or functional disturbances. For example, a variety of diseases affecting neurologic function, including diabetes mellitus and multiple sclerosis, may interrupt sexual arousal. In addition, estrogen deficiency, causing vaginal atrophy, is a common cause of sexual dysfunction. For a discussion of other causes of female sexual dysfunction, see, e.g., Kaplan, The Evaluation of Sexual Disorders: Psychological and Medical Aspects (New York: Brunner-Mazel, 1983), and Kolodny et al., Textbook of Sexual Medicine (Boston, Mass.: Little, Brown & Co., 1979).

The present invention specifically pertains to those women who suffer from or are prone to dyspareunia. The phrase “women who suffer from dyspareunia”, is intended to include women who experience pain and discomfort during sexual intercourse, often in combination with post-coital vaginal burning, vaginal dryness, pelvic aching, urinary discomfort, and/or lower abdominal distress. Painful intercourse, sometimes coupled with vaginal burning and/or irritation during urination, are the most typical manifestations of dyspareunia; see Masters and Johnson, Human Sexual Response, cited supra. Dyspareunia is thought to affect approximately 40% of women, and it has been estimated that over 40 million women will suffer dyspareunia at some time in their lives. On the order of twenty-five million will experience dyspareunia in the peri- and postmenopausal period (see Kelly, S. (1992) Clinical Practice and Sexuality 8(8):2 and Sato et al. (1992) Clinical Practices in Sexuality 8(5):1). Contemporary symptomatic treatments generally involve the use of physiologically safe lubricants such as egg white, K-Y surgical lubrication jelly (hydroxyethyl-cellulose), and products sold under the brand names Astroglide®, and Replens®. See, for example, Semmens (1974) Medical Aspects of Human Sexuality 8:85-86, and Frishmen et al. (1992) Fertility and Sterility 58(3):630. When symptomatic treatment fails, pharmacological treatment may be indicated.

Estrogen therapy is commonly used in the pharmacological treatment of any or all of the various symptoms of dyspareunia. Estrogen-based therapies are generally used to increase mucous production, provide vasodilatory effects, or to increase the general health of the vagina. Nadelson et al., eds., Treatment Interventions in Human Sexuality (New York: Plenum Press, 1983). In such treatments, estrogen is administered orally, parenterally (e.g., by injection), or topically. With oral administration, the estrogen concentration encountered by the liver is generally four- to five-fold greater than estrogen levels in peripheral blood (the “first pass effect”). This effect may lead to an undesirable increase in the production of certain coagulation factors and renin substrates by the liver. Parenterally administered estrogen avoids the first pass effect in the liver. However, all estrogen-based therapies are known to increase the risk of endometrial hyperplasia endometrial cancer and breast cancer in treated individuals.

Because of the increased risk of endometrial hyperplasia and endometrial cancer encountered with unopposed estrogen therapies, estrogen/progestogen combinations have been employed. However, progestogens are known to have some androgenic activity. Further, common side effects from such therapies include uterine bleeding and the continuation of menstrual periods. Accordingly, there remains a need in the art to provide safer and more ways of treating women who are suffering from dyspareunia.

The present invention is directed to the aforementioned need in the art, and provides a new, highly effective method of treating dyspareunia. The method involves topical administration of a pharmaceutical formulation containing a vasoactive agent, in particular the nitric oxide-releasing agent nitroglycerin. The method is unexpectedly advantageous in eliminating or at least substantially minimizing the number and severity of symptoms in a woman suffering from dyspareunia.

### SUMMARY OF THE INVENTION

One aspect of the invention relates to a method for treating a woman suffering from dyspareunia by topically administering a pharmaceutical formulation containing a therapeutically effective amount of nitroglycerin, wherein “topical” administration is to the vulvar region (i.e., the clitoris as well as the immediately surrounding region) or to the vagina, or both.

Another aspect of the invention pertains to pharmaceutical formulations useful in conjunction with the aforementioned method.

Yet another aspect of the invention relates to a kit for a female individual to use in carrying out the aforementioned method to self-administer a nitroglycerin formulation contained within the kit.

In one aspect of the invention, then, a method is provided for treating a woman suffering from dyspareunia by topically applying a pharmaceutical formulation containing a therapeutically effective amount of nitroglycerin. Nitroglycerin may or may not be the only active agent in the formulation. That is, the formulation may contain one or more other active agents in addition to the nitroglycerin. Like nitroglycerin, such secondary active agents are typically vasoactive agents, preferably vasodilators. Preferred vasodilators include, by way of example, naturally occurring prostaglandins, synthetic prostaglandin derivatives, endothelial-derived relaxation factors, vasoactive intestinal polypeptide agonists, smooth muscle relaxants, leukotriene inhibitors, and phosphodiesterase (PDE) inhibitors, including inhibitors of Type III PDE, Type IV PDE, and Type V PDE, as well as non-selective PDE inhibitors. Other preferred additional active agents include steroids such as androgens, estrogens and progestins, as well as selective androgen receptor modulators and selective estrogen receptor modulators. The secondary active agents are not necessarily incorporated in the nitroglycerin formulation, but may be administered in separate formulations or dosage forms, either simultaneously or at different times. Any number of drug delivery platforms may be used, e.g., liquid and semi-solid solutions, creams, gels, lotions, suspensions, ointments, pastes, foams, patches, suppositories, bioadhesive films, and so forth.

In another aspect of the invention, a topical pharmaceutical formulation is provided for carrying out the aforementioned method. The formulation contains a therapeutically effective amount of nitroglycerin and a pharmaceutically acceptable carrier suitable for vulvar and/or vaginal administration, and, optionally, one or more additional pharmacologically active agents. Examples of preferred formulations are liquid and semi-solid solutions, creams, and bioerodible adhesive films.

In still another aspect of the invention, a packaged kit is provided for a patient to carry out the aforementioned method in the self-administration of the nitroglycerin formulation. The packaged kit includes: the formulation per se, e.g., as a dosage form containing a therapeutically effective amount of nitroglycerin in combination with a pharmaceutically acceptable topical carrier; a container housing the formulation during storage and prior to administration; and instructions, e.g., written instructions on a package insert or label, for carrying out drug administration in a therapeutically effective manner to treat dyspareunia. A suitable dosage form may be any of those described herein, preferably containing a unit dosage of nitroglycerin, the unit dosage being a therapeutically effective dosage for treatment of dyspareunia.

Yet another aspect of the invention relates to a biodegradable adhesive film for application to the vulvar region of a female patient suffering from dyspareunia, which comprising a therapeutically effective amount of nitroglycerin in a biodegradable adhesive polymer composition effective to provide for sustained drug release.

### DETAILED DESCRIPTION OF THE INVENTION

I. Definitions and Overview

Before describing the present invention in detail, it is to be understood that this invention is not limited to delivery of specific drugs, carriers or use of particular drug delivery systems, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, reference to “an active agent” includes a single active agent as well as a combination or mixture of two or more active agents, reference to “a pharmaceutically acceptable carrier” includes a single pharmaceutically acceptable carrier as well as a combination or mixture of two or more different pharmaceutically acceptable carriers, and the like.

In describing and claiming the present invention, the following terminology will be used in accordance with the definitions set out below.

The terms “active agent,” “pharmacologically active agent” and “drug” are used interchangeably herein to refer to a chemical compound that induces a desired pharmacological, physiological effect, i.e., in this case, treatment of dyspareunia. The terms also encompass pharmaceutically acceptable, pharmacologically active derivatives of those active agents specifically mentioned herein, including, but not limited to, salts, esters, amides, prodrugs, active metabolites, inclusion complexes, analogs, and the like. When the terms “active agent,” “pharmacologically active agent” and “drug” are used, then, or when an active agent such as nitroglycerin or a secondary active agent such as a prostaglandin or a phosphodiesterase inhibitor is specifically identified, it is to be understood that applicants intend to include the active agent per se as well as pharmaceutically acceptable, pharmacologically active salts, esters, amides, prodrugs, active metabolites, inclusion complexes, analogs, etc.

By “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical formulation administered to a patient without causing any undesirable biological effects or interacting in a deleterious manner with any of the other components of the formulation in which it is contained. “Pharmacologically active” (or simply “active”) as in, for example, a “pharmacologically active” derivative or metabolite, refers to a derivative or metabolite having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well, i.e., therapeutically effective to enhance female sexual desire and responsiveness.

The term “carriers” (or “vehicles”) as used herein refers to conventional carrier materials that are suitable for incorporation into a pharmaceutical formulation for vulvar and/or vaginal drug administration, and that are “pharmaceutically acceptable” as defined herein.

By an “effective” amount or a “therapeutically effective amount” of a drug or pharmacologically active agent is meant a nontoxic but sufficient amount of the drug or agent to provide the desired effect, i.e., treatment of dyspareunia. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation. Furthermore, the exact “effective” amount of an active agent incorporated into a composition or dosage form of the invention is not critical, so long as the amount of the active agent delivered is within a therapeutically effective range.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, “treatment” of dyspareunia, as the term is used herein, encompasses both prevention of dyspareunia in clinically asymptomatic individuals who are prone to or likely to develop dyspareunia as well as treatment of an individual exhibiting symptoms of dyspareunia.

The term “topical” delivery or administration is used to refer to direct administration of a pharmaceutical formulation to the vulvar area of the individual undergoing treatment, or to the individual's vagina. Vaginal delivery of a pharmaceutical formulation herein typically involves administration to the distal several centimeters of the vagina, while administration to the “vulvar area” generally encompasses direct application to the clitoris and/or the surrounding vulvar area.

The term “unit dosage form” refers to a physically discrete unit containing a single, unitary dosage administration to a human subject, each unit dosage representing a predetermined quantity of an active agent effective to produce the desired therapeutic effect.

The term “controlled release” is intended to refer to any drug-containing formulation in which release of the drug is not immediate, i.e., with a “controlled release” formulation, oral administration does not result in immediate release of the drug into an absorption pool. The term is used interchangeably with “nonimmediate release” as defined in Remington: The Science and Practice of Pharmacy, 20^{th} edition (Lippincott Williams & Wilkins, 2000). As discussed therein, immediate and nonimmediate release can be defined kinetically by reference to the following equation:

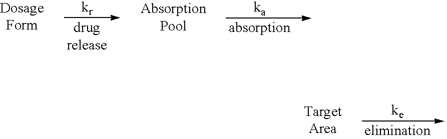

The “absorption pool” represents a solution of the drug administered at a particular absorption site, and kᵣ, kₐ and kₑ are first-order rate constants for (1) release of the drug from the formulation, (2) absorption, and (3) elimination, respectively. For immediate release dosage forms, the rate constant for drug release kᵣ is far greater than the absorption rate constant kₐ. For controlled release formulations, the opposite is true, i.e., kᵣ<<kₐ, such that the rate of release of drug from the dosage form is the rate-limiting step in the delivery of the drug to the target area. The term “controlled release” as used herein includes any nonimmediate release formulation, including but not limited to sustained release, delayed release and pulsatile release formulations.

The term “sustained release” is used in its conventional sense to refer to a drug formulation that provides for gradual release of a drug over an extended period of time, and that preferably, although not necessarily, results in substantially constant blood levels of a drug over an extended time period. A sustained release formulation may be administered once to provide a single bolus dose of the drug, which is then effective for up to a day or even up to several days.

Accordingly, the invention relates to a method for treating a woman suffering from dyspareunia, and involves topical administration of a pharmaceutical formulation containing a therapeutically effective amount of nitroglycerin, a pharmaceutically acceptable carrier, and, optionally, one or more additional active agents such as vasoactive agents, particularly vasodilators. In a unit dosage form, the “unit dosage” of nitroglycerin that is administered in a single application is in the range of about 0.05 mg to about 150 mg, typically about 0.1 mg to about 30 mg, preferably about 0.2 mg to about 20 mg, and most preferably about 0.2 mg to about 10 mg.

In a solution, gel, cream, lotion, suspension, ointment, paste, foam, patch, suppository, or the like, the composition will contain a sufficient concentration of nitroglycerin such that the aforementioned therapeutically effective amount of nitroglycerin is delivered by application of about 0.1 g to 1.0 g of the composition. With vaginal suppositories, or with suppositories applied directly to the vulvar region, a total suppository weight in the range of about 0.1 g to 0.5 g is common, again, meaning that the therapeutically effective unit dosage of nitroglycerin is delivered in a suppository having a total weight within the aforementioned range.

The formulation is applied topically to a female individual suffering from or prone to dyspareunia, as noted above, wherein “topical” application means that the formulation is administered to the individual's clitoris or other area in the vulvar region, and/or the distal several centimeters of the vagina. The patient population treated using the method of the invention, i.e., women “suffering from” or “prone to” dyspareunia, are women who experience or are predisposed to experience pain and discomfort during sexual intercourse as well as possible additional symptoms such as post-coital vaginal burning, pelvic aching, urinary discomfort, lower abdominal distress, and vaginal dryness. Dyspareunia, as the term is used herein, does not include primary or secondary anorgasmia, decreased intensity of or pleasure in orgasms, or substance-induced sexual dysfunction.

II. Pharmaceutical Formulations

Generally, formulations of the invention will contain about 0.001 wt % to about 15.0 wt % nitroglycerin, preferably about 0.01 wt % to about 10.0 wt %, more preferably about 0.1 wt % to about 5.0 wt % nitroglycerin, and most preferably about 0.3 wt % to about 3.0 wt % nitroglycerin. The topical nitroglycerin-containing formulation used in conjunction with the method of the invention contains one or more pharmaceutically acceptable carriers suited to the particular type of formulation, i.e., solution, gel, cream, lotion, ointment, suppository, or the like. The carriers are comprised of materials of naturally occurring or synthetic origin that do not adversely affect the active agent(s) or any other components of the formulation. Depending on the type of formulation, suitable carriers typically include water, silicone, waxes, petroleum jelly, polyethylene glycol, propylene glycol, liposomes, sugars such as mannitol and lactose, and a variety of other materials, again depending, on the specific type of formulation employed.

The formulations may also include a chemical compound to enhance permeation of the active agent through the mucosal tissue and skin, i.e., a “permeation enhancer.” Suitable permeation enhancers include those generally useful in conjunction with topical, transdermal or transmucosal drug delivery. Examples of suitable permeation enhancers include the following: sulfoxides such as dimethylsulfoxide (DMSO), decylmethylsulfoxide (C₁₀MSO), and 2-alkyl-(tetrahydrothiophene)-1-oxides; ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin (U.S. Pat. No. 4,783,450 to Fawzi, et al.); the 1-substituted azacycloheptan-2-ones, particularly 1-hexyl azacycloheptan-2-one, 1-octyl azacycloheptan-2-one, 1-decyl azacycloheptan-2-one, and 1-dodecyl azacycloheptan-2-one (laurocapram; Azone®); lower molecular weight alcohols such as ethanol, propanol, octanol, decanol, benzyl alcohol, and the like; fatty alcohols such as behenyl alcohol, cetyl alcohol, elaidyl alcohol, erucyl alcohol, isostearyl alcohol, lauryl alcohol, myristyl alcohol, oleyl alcohol, palmitoleyl alcohol, petroselinyl alcohol, and stearyl alcohol; fatty acids such as capric acid, lauric acid, oleic acid, stearic acid and valeric acid; fatty acid esters such as isopropyl myristate, isopropyl palmitate, methylpropionate, cetyl lactate, myristyl lactate, lauryl lactate, isostearyl lactate, ethyl lactate, ethyl oleate, ethyl linoleate, and isopropyl linoleate; polyols and esters thereof such as propylene glycol, ethylene glycol, glycerol, butanediol, polyethylene glycol, and polyethylene glycol monolaurate (PEGML; see, e.g., U.S. Pat. No. 4,568,343 to Leeper, et al.); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly salicylic acid, citric acid and succinic acid. Mixtures of two or more enhancers may also be used.

Some of the aforementioned compounds may serve one or more secondary purposes, i.e., in addition to enhancing the flux of the applied active agent through the body surface at the treatment area. For example, isopropyl myristate and other fatty acid esters are nonionic surfactants that act not only as penetration enhancers, but also as emollients and solubilizers. As another example, ethanol and other alcohols act as disinfectants, antimicrobial agents and solubilizers, in addition to serving as penetration enhancers.

The formulations of the invention may take the form of solutions, gels, creams, lotions, suspensions, ointments, pastes, foams, patches, suppositories, etc., and/or may be prepared so as to contain liposomes, micelles, and/or microspheres, and the like. Alternatively, the formulations may be contained within a vaginal ring (e.g., as disclosed in U.S. Pat. No. 5,188,835 to Lindskog et al.), or within a tampon, suppository, sponge, pillow, puff, or osmotic pump system; these latter platforms are useful solely for vaginal delivery.

Liquid and semi-solid formulations, e.g., solutions, gels, creams, lotions, suspensions, ointments, pastes, foams, etc., for use in conjunction with the invention will contain at least one liquid or semi-solid carrier, and preferably more than one such carrier, with the carriers singly or in combination providing for solubilization of the active agent(s). It is also desirable that at least one of the carriers used enhance penetration of the active agent into the patient's body surface following topical application of the formulation. As noted above, carriers such as ethanol and other alcohols are useful not only as solubilizers and penetration enhancers, but also as disinfectants and antimicrobial preservatives, while nonionic surfactants such as fatty acid esters can serve as emollients and as well as solubilizers and penetration enhancers.

Examples of suitable carriers for use in the aforementioned formulations, then, include, but are not limited to:

fatty acids, e.g., arachidic acid (n-eicosanoic acid), arachidonic acid, behenic acid (docosanoic acid), capric acid (n-decanoic acid), caproic acid (n-hexanoic acid), caproleic acid (9-decenoic acid), caprilic acid (n-octanoic acid), docosadienoic acid, docosahexaenoic acid, docosapentaenoic acid, eicosadienoic acid, eicosahexaenoic acid, eicosapentaenoic acid, eicosatrienoic acid, elaidic acid (trans-9-octadecanoic acid), eleosteroic acid, erucic acid (13-docosenoic acid), heneicosanoic acid, heptacosanoic acid, heptadecanoic acid, heptanoic acid, hexacosanoic acid, isostearic acid, lauric acid (n-dodecanoic acid), lignoceric acid (n-tetracosanoic acid), linoleic acid, α-linolenic acid, γ-linolenic acid, myristic acid (n-tetradecanoic acid), myristoleic acid, neodecanoic acid, nervonic acid (cis-15-tetracosenoic acid), nonacosanoic acid, nonadecanoic acid, octacosanoic acid, oleic acid, palmitic acid (n-hexadecanoic acid), palmitoleic acid, pelargonic acid (nonanoic acid), pentadecanoic acid, pentacosanoic acid, petroselenic acid, phytanic acid, stearic acid (n-octadecanoic acid), triacontanoic acid, tricosanoic acid, tridecanoic acid, undecanoic acid, and vaccenic acid, with C₁₀-C₁₈ fatty acids such as capric, lauric and oleic acids preferred;

fatty alcohols that derive from the fatty acids above, i.e., the terminal carboxylic acid group COOH of the fatty acid is replaced with a CH₂OH group, including, by way of example, behenyl alcohol, cetyl alcohol, elaidyl alcohol, erucyl alcohol, isostearyl alcohol, lauryl alcohol, myristyl alcohol, oleyl alcohol, palmitoleyl alcohol, petroselinyl alcohol, and stearyl alcohol;

bile acids such as cholic acid, deoxycholic acid, lithocholic acid, chenodeoxycholic acid (also referred to as “chenodiol” or “chenic acid”), ursodeoxycholic acid, taurocholic acid, taurodeoxycholic acid, taurolithocholic acid, taurochenodeoxycholic acid, tauroursodeoxycholic acid, glycocholic acid, glycodeoxycholic acid, glycolithocholic acid, glycochenodeoxycholic acid, and glycoursodeoxycholic acid, as well as corresponding salts thereof;

fatty acid esters and fatty esters of alcohols, e.g., cetyl lactate, myristyl lactate, lauryl lactate, isostearyl lactate, and stearyl lactate, ethyl lactate, isopropyl myristate, isopropyl palmitate, ethyl linoleate, isopropyl linoleate, methyl laurate, ethyl oleate, isopropyl n-decanoate, isopropyl myristate, isopropyl palmitate, sucrose monooleate, cholesterol stearate, octyldodecyl myristate, propylene glycol dilaurate, propylene glycol monooleate, propylene glycol dioctanoate, propylene glycol dicaprylate, propylene glycol dicaprate, glycerol monolaurate, glycerol monooleate, glycerol monostearate; the sorbitan fatty acid esters sorbitan monopalmitate, sorbitan monooleate, sorbitan dioleate, sorbitan trioleate, sorbitan sesquioleate, sorbitan isostearate, sorbitan diisostearate, sorbitan tristearate, and sorbitan monolaurate; and the sucrose fatty acid esters sucrose monooleate, sucrose monostearate, sucrose monolaurate, sucrose distearate, sucrose dipalmitate, and sucrose monopalmitate; and

polyoxyalkylene fatty acid esters and polyoxyalkylene sorbitan fatty acid esters, such as polyoxyethylene and polyoxypropylene glyceryl stearate, laurate, and palmitate, e.g., polyethylene glycol (PEG)-20 glyceryl stearate, polypropylene glycol PPG-10 glyceryl stearate, PEG-15 glyceryl laurate, PEG-20 glyceryl laurate, PEG-30 glyceryl laurate, PEG-40 sorbitol septaoleate, PEG-40 glyceryl laurate, polyoxyethylene sorbitan monolaurate (for example, PEG-20 sorbitan monolaurate, available commercially under the tradename Tween®20), polyoxyethylene sorbitan monopalmitate (for example, PEG-20 sorbitan monopalmitate, available commercially under the tradename Tween®40), polyoxyethylene sorbitan monostearate (for example, PEG-20 sorbitan monostearate, available commercially under the tradename Tween®60), polyoxyethylene sorbitan monooleate (for example, PEG-20 sorbitan monooleate, available commercially under the tradename Tween®80), polyoxyethylene sorbitan trioleate (e.g., PEG-20 sorbitan trioleate), polyoxyethylene sorbitol septaoleate, polyoxyethylene sorbitan monooleate, polyoxypropylene sorbitan monooleate, polyoxypropylene sorbitan monopalmitate, polyoxypropylene sorbitan trioleate, and polyoxypropylene sorbitol septaoleate.

Additional components that are preferred additives in liquid and semi-solid formulations are silicones such as dimethicone, phenyl dimethicone, cyclic silicones such as the cyclomethicones (e.g., Cyclomethicone DC344, obtained from Dow Corning), and silicone esters such as diisostearoyl trimethylolpropane, all of which serve as emollients and improve the skin “feel” of the formulation. In some cases, the formulations may include an enzyme inhibitor, i.e., a compound effective to inhibit enzymes present in the vagina or vulvar area that could degrade or metabolize the active agent. That is, inhibitors of enzymes that decrease or eliminate the activity of the active agent may be included in the formulation so as to effectively inhibit the action of those enzymes. For example, inhibitors of prostaglandin-degrading enzymes may be included if a prostaglandin is present as a secondary agent. Such inhibitors include, for example, fatty acids, fatty acid esters, and NAD inhibitors.

Gels: The active agent can also be incorporated into a gel formulation using known techniques. Two-phase gel systems generally comprise a suspension or network of small, discrete particles interpenetrated by a liquid to provide a dispersed phase and a liquid phase. Single-phase gel systems are formed by distributing organic macromolecules uniformly throughout a liquid such that there are no apparent boundaries between the dispersed and liquid phases. Suitable gelling agents for use herein include synthetic macromolecules (e.g., Carbomers, polyvinyl alcohols and polyoxyethylene-polyoxypropylene copolymers), gums such as tragacanth, as well as sodium alginate, gelatin, methylcellulose, sodium carboxymethylcellulose, methylhydroxyethyl cellulose and hydroxyethyl cellulose. In order to prepare a uniform gel, dispersing agents such as alcohol or glycerin can be added, or the gelling agent can be dispersed by trituration, mechanical mixing or stirring, or combinations thereof. The concentration of nitroglycerin in a gel formulation herein will be the same as that described above with respect to a solution formulation, and will likewise apply to the other topical formulations described infra.

Lotions: Lotions are preparations that may be applied without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. It is generally necessary that the insoluble matter in a lotion be finely divided. Lotions will typically contain suspending agents to produce better dispersions as well as compounds useful for localizing the active agent in contact with the skin, e.g., methylcellulose, sodium carboxymethylcellulose, or the like.

Emulsions: Pharmaceutical emulsion formulations are generally formed from a dispersed phase (e.g., the nitroglycerin and any additional pharmacologically active agent), a dispersion medium and an emulsifying agent. If desired, emulsion stabilizers can be included in the formulation as well. A number of pharmaceutically useful emulsions are known in the art, including oil-in-water (o/w) formulations, water-in-oil (w/o) formulations and multiple emulsions such as w/o/w or o/w/o formulations. Emulsifying agents suitable for use in such formulations include, but are not limited to, TWEEN®60, Span 80®, cetostearyl alcohol, myristyl alcohol, glyceryl monostearate and sodium lauryl sulfate.

Ointments: Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, see Remington for further information.

Creams: Creams, as also well known in the art, are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Pastes: Pastes are semisolid dosage forms in which the active agent is suspended in a suitable base. Depending on the nature of the base, pastes are divided between fatty pastes or those made from a single-phase aqueous gel. The base in a fatty paste is generally petrolatum or hydrophilic petrolatum or the like. The pastes made from single-phase aqueous gels generally incorporate carboxymethylcellulose or the like as a base.

Formulations may also be prepared with liposomes, micelles, and microspheres. Liposomes are microscopic vesicles having a lipid wall comprising a lipid bilayer, and can be used as drug delivery systems herein as well. Generally, liposomal formulations include cationic, anionic and/or neutral liposomes. Cationic liposomes are readily available. For example, N[1-2,3-dioleyloxy)propyl]-N,N,N-triethylammonium (DOTMA) liposomes are available under the tradename Lipofectin® (GIBCO BRL, Grand Island, N.Y.). Similarly, anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), and dioleoylphoshatidyl ethanolamine (DOPE), among others. These materials can also be mixed with DOTMA in appropriate ratios. Methods for making liposomes using these materials are well known in the art.

Micelles are known in the art as comprised of surfactant molecules arranged so that their polar headgroups form an outer spherical shell, while the hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethyl-ammonium chloride, dodecylammonium chloride, polyoxyl 8 dodecyl ether, polyoxyl 12 dodecyl ether, nonoxynol 10 and nonoxynol 30. Micelle formulations can be used in conjunction with the present invention either by incorporation into the reservoir of a topical or transdermal delivery system, or into a formulation to be applied to the body surface.

Microspheres, similarly, may be incorporated into the present formulations and drug delivery systems. Like liposomes and micelles, microspheres essentially encapsulate a drug or drug-containing formulation. Microspheres are generally, although not necessarily, formed from synthetic or naturally occurring biocompatible polymers, but may also be comprised of charged lipids such as phospholipids. Preparation of microspheres is well known in the art and described in the pertinent texts and literature.

Vaginal suppositories are typically manufactured with polyethylene glycol (PEG), polyethylene oxide and/or other low melting point or water-soluble polymers including fatty acid esters. Suppositories may also be applied to the vulvar region, in which case these dosage forms, which are solid at ambient temperature, rapidly melt when placed on the clitoris and within the surrounding vulvar region. Alternatively, or in addition, the suppository may be administered vaginally.

Methods of preparing such formulations are known, or will be apparent, to those skilled in this art; for example, see Remington: The Science and Practice of Pharmacy, 20^{th} edition (Lippincott Williams & Wilkins, 2000).

The above pharmaceutical formulations are formed by dispersing or dissolving the active agent, in particulate form, uniformly throughout the carrier or base using conventional techniques, typically by levigating the agent with a small quantity of the carrier or base to form a concentrate, which is then diluted with further base. Alternatively, a mechanical mixer may be used. Creams, lotions and emulsions are typically formed using a two-phase heat system, wherein the components of the oil phase are combined under heat to provide a liquified, uniform system. The aqueous phase components are separately combined, also using heat. The oil and aqueous phases are then admixed with constant agitation and allowed to cool. At this point, concentrated agents may be added as a slurry. Volatile or aromatic materials can be added after the emulsion has sufficiently cooled.

The pharmaceutical formulations of the invention are typically contained within drug delivery systems that provide a specific, predetermined agent release profile, e.g., immediate release, sustained release, immediate release of an initial drug “burst” followed by sustained (e.g., steady state) release, pulsatile release, or cyclical release. Such systems can include, for example, osmotic pumps that are capable of delivering variable amounts of the agent in a pulsatile manner. Osmotic pump systems typically involve incorporation of the pharmaceutical agent within a hard coating shell that is usually semi-permeable, e.g., a microporous cellulose acetate latex coating; the shell further contains a bore hole drilled into the outer layer. After delivery, water from the surrounding moist environment is osmotically pulled into shell through the bore hole, dissolving the agent and creating a high integral pressure sufficient to effect release of the agent from the shell. In this manner, the active agent is automatically “pulsed” out of the delivery system. A number of suitable osmotic pumps have been described in the art. See, for example, Appel et al. (1992) Pharm. Res. 9:1664-1667 and Kleinbloesem et al. (1984) Clin. Pharm. Therapeut. 36:396-401.

Other topically administrable drug delivery formulations and systems capable of providing a controlled release (e.g., sustained release) profile include those in which the active agent is contained within a matrix of a gradually bioerodible (e.g., hydrolyzable or otherwise degradable) polymer. Such polymers are generally selected such that they bioerode in the presence of moisture, such as that emanating from an individual's skin or mucosal surface, and provide for sustained agent release at readily predictable rates. Examples of such polymers include, without limitation, crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, cream, paste, or the like, dispersing agents such as alcohol or glycerin can be added, or the gelling agent can be dispersed by trituration, mechanical mixing, stirring, or combinations thereof.

More particularly, release of the active agent can be controlled by dissolution (bioerosion) of a polymer using either encapsulated dissolution control or matrix dissolution control. In encapsulated dissolution control, the active agent is coated with a membrane of slowly dissolving polymeric or wax materials. When the encapsulating membrane has dissolved, the agent core is available for immediate release and adsorption across the epithelial or mucosal surfaces of the vagina or vulvar area. Bioerodible coating materials may be selected from a variety of natural and synthetic polymers, depending on the agent to be coated and the desired release characteristics. Exemplary coating materials include gelatins, carnauba wax, shellacs, ethylcellulose, cellulose acetate phthalate and cellulose acetate butyrate. Release of the agent is controlled by adjusting the thickness and dissolution rate of the polymeric membrane. A uniform sustained release formulation can be attained by compressing a population of particles of the agent with varying membrane thickness (e.g., varying erosion times) into a tablet form for a single administration.

In matrix dissolution control, the active agent is dissolved or dispersed within a matrix of, for example, an erodible wax. The agent is released for adsorption across the epithelial or mucosal surfaces of the vagina or vulvar area as the matrix bioerodes. The rate of agent availability is generally controlled by the rate of penetration of the dissolution media (i.e., vaginal fluids) into the matrix, wherein the rate of penetration is dependent on the porosity of the matrix material. Bioerodible matrix dissolution delivery systems can be prepared by compressing the active agent with a slowly soluble polymer carrier into a tablet or suppository form. There are several methods of preparing drug/wax particles including congealing and aqueous dispersion techniques. In congealing methods, the active agent is combined with a wax material and either spray-congealed, or congealed and then screened. For an aqueous dispersion, the active agent/wax combination is sprayed or placed in water and the resulting particles collected. Matrix dosage formulations can be formed by compaction or compression of a mixture of nitroglycerin, any secondary active agent(s), polymer and excipients.

In an alternative embodiment, the pharmaceutical formulation is administered in the form of a biodegradable adhesive film or sheet that adheres to the vulvar area. Such drug delivery systems are generally composed of a biodegradable adhesive polymer based on a polyurethane, a poly(lactic acid), a poly(glycolic acid), a poly(ortho ester), a polyanhydride, a polyphosphazene, or a mixture or copolymer thereof. Preferred biodegradable adhesive polymers include polyurethanes and block copolyurethanes containing peptide linkages, simple mixtures of polyurethanes and polylactides, and copolymers of acrylates and mono- or disaccharide residues.

Delivery of an “as-needed” or “on-demand” dose of nitroglycerin with topical formulations intended for application to the vulvar region, and/or with vaginal suppositories, may be effected by using a carrier and any excipients effective to provide for immediate release of the active agent from the formulation or dosage form. Suitable techniques and practical considerations in formulating an immediate release topical formulation will be known to those in the art and are available by reference to the pertinent texts and literature. See, e.g., Addicks et al., “Drug Delivery from Topical Formulations: Theoretical Prediction and Experimental Assessment,” in Topical Drug Delivery Formulations, eds. Osborne et al. (Marcel Dekker, 1990). Generally, the total concentration Cs of drug in the formulation should be much higher than the solubility Q of the drug in the formulation, typically at least 50% higher, preferably at least 75% higher. Further, a carrier can be selected such that the active agent's affinity for the carrier is lower than its affinity for the treated body surface. Suitable carriers can be determined by testing, using routine procedures, a series of different carriers containing the active agent and selecting those carriers that provide the greatest flux of the active agent to the intended tissue, e.g., clitoral tissue. Additionally, one or more permeation enhancers (as discussed above) and/or detergents may be incorporated into the formulation to ensure a rate of delivery sufficient for on-demand administration. A combination of these approaches as well as other approaches may be used to effect delivery of an on-demand dose.

Once the initial, on-demand dose is delivered, the drug delivery system, if present, and/or any remaining formulation may be removed or may remain in place, depending on the preferences of the individual. Alternatively, the formulation and optional drug delivery system may be designed to provide both initial “on-demand” release of the active agent, i.e., as a single, bolus dose, as well as sustained release thereafter, e.g., pulsatile, continuous or cyclical drug release. Such systems can include, for example, osmotic release systems as discussed above, providing that they are capable of delivering an initial, on-demand release of the active agent in addition to variable amounts of the agent in a pulsatile or sustained release manner thereafter.

III. Secondary Active Agents

In addition to the therapeutically effective amount of nitroglycerin, the formulations of the invention may contain one or more secondary active agents. Preferred secondary active agents enhance the therapeutic efficacy of the nitroglycerin, or are themselves useful in the treatment of dyspareunia.

Secondary active agents include vasoactive agents, particularly vasodilators. Preferred vasodilators are selected from the group consisting of vasoactive prostaglandins, vasoactive intestinal polypeptide (VIP) and analogs and agonists thereof, endothelin-derived relaxation factors, smooth muscle relaxants, leukotriene inhibitors, pharmaceutically acceptable, pharmacologically active salts, esters, amides, prodrugs, active metabolites, inclusion complexes, analogs, etc, and combinations of any of the foregoing. Other suitable secondary agents include phosphodiesterase inhibitors, rho kinase inhibitors, melanocortin peptides, endothelin antagonists, growth factors and other peptidyl drugs; selective androgen receptor modulators (SARMs), selective estrogen receptor modulators (SERMs), neuropeptides, amino acids, serotonin agonists, serotonin antagonists, calcium channel blockers, potassium channel openers, potassium channel blockers, dopamine agonists, dopamine antagonists, steroid hormones, steroid antagonists and partial agonists, and combinations of any of the foregoing.

Particularly preferred vasoactive agents suitable as secondary active agents herein are vasoactive prostaglandins selected from the group consisting of naturally occurring prostaglandins, semisynthetic prostaglandins, synthetic prostaglandins, and pharmaceutically acceptable, pharmacologically active salts, esters, amides, inclusion complexes, prodrugs, metabolites, and analogs thereof. Racemic, optically enriched or purified stereoisomers of any of these compounds are also included. A suitable unit dose of a prostaglandin herein is in the range of approximately 1 to 5000 μg, preferably in the range of approximately 20 to 2000 μg. Preferred prostaglandins include, but are not limited to, the naturally occurring prostaglandins prostaglandin E₀ (PGE₀, also referred to 13,14-dihydro-PGE₁; hereinafter, the abbreviation “PG” is used for “prostaglandin”), PGE₁, 19-hydroxy-PGE₁, PGE₂, 19-hydroxy-PGE₂, PGA₁, 19-hydroxy-PGA₁, PGA₂, 19-hydroxy-PGA₂, PGB₁, 19-hydroxy-PGB₁, PGB₂, 19-hydroxy-PGB₂, PGB₃, PGD₂, PGF_{1α}, PGF_{2α}(dinoprost), PGE₃, PGF_{3α}, PGI₂ (prostacyclin), and combinations thereof. PGE₀, PGE₁, PGE₂, and the hydrolyzable lower alkyl esters thereof (e.g., the methyl, ethyl and isopropyl esters) are, however, particularly preferred. Other suitable prostaglandins are exemplified, without limitation, by arboprostil, carbaprostacyclin, carboprost tromethamine, dinoprost tromethamine, dinoprostone, enprostil, iloprost, lipoprost, gemeprost, metenoprost, sulprostone, tiaprost, viprostil (CL 115,347), viprostil methyl ester, 16,16-dimethyl-Δ²-PGE₁ methyl ester, 15-deoxy-16-hydroxy-16-methyl-PGE₁ methyl ester (misoprostol), 16,16-dimethyl-PGE₁, 11-deoxy-15-methyl-PGE₁, 16-methyl-18,18,19,19-tetrahydrocarbacyclin, 16(RS)-15-deoxy-16-hydroxy-16-methyl-PGE₁ methyl ester, (+)-4,5-didehydro-16-phenoxy-α-tetranor-PGE₂ methyl ester, 11-deoxy-11α,16,16-trimethyl-PGE₂, (+)-11α,16α,16β-dihydroxy-1,9-dioxo-1-(hydroxymethyl)-16-methyl-trans-prostene, 9-chloro-16,16-dimethyl-PGE₂, 16,16-dimethyl-PGE₂, 15(S)-15-methyl-PGE₂, 9-deoxy-9-methylene-16,16-dimethyl-PGE₂, 19(R)-hydroxy-PGE₂, and 11-deoxy-16,16-dimethyl-PGE₂.

Still other vasoactive agents include vasoactive intestinal polypeptide (VIP), VIP agonists, pharmaceutically acceptable, pharmacologically active salts, esters, amides, prodrugs, active metabolites, inclusion complexes, analogs, etc, and combinations of any of the foregoing. The sequence of human VIP, which is the same as rat, bovine and porcine known in the art. VIP sequences from other species are known to exhibit homology to human VIP and therefore expected to exhibit VIP agonistic and/or antagonistic activity. Partial agonists of VIP that are also antagonists of VIP, for example, agonists that are less active than endogenously secreted VIP may be used in the methods of the invention depending upon activity relative to physiologic human VIP and local dosage. The usefulness of partial VIP agonists for practicing the invention may be ascertained by conventional trials and pharmacologic assays known in the art. Preferred VIP analogs and derivatives are hydrolyzable lower alkyl esters of VIP per se. Specific VIP analogs and agonists useful in conjunction with the present invention are described in U.S. Patent Application Publication 2002/0099003 to Wilson et al.

Additional vasoactive agents useful as secondary active agents herein include endothelin-derived relaxation factors (“EDRFs”) such as nitric oxide releasing agents other than nitroglycerinper se, e.g., sodium nitroprusside and diazenium diolates, or “NONOates.” NONOates include, but are not limited to, (Z)-1-{N-methyl-N-[6-(N-methyl-ammoniohexyl)amino]} diazen-1-ium-1,2-diolate (“MAHMAINO”), (Z)-1-[N-(3-ammoniopropyl)-N-(n-propyl)amino]-diazen-1-ium-1,2-diolate (“PAPA/NO”), (Z)-1-{N-[3-aminopropyl]-N-[4-(3-aminopropylammonio)butyl]amino} diazen-1-ium-1,2-diolate (spermine NONOate or “SPER/NO”) and sodium (Z)-1-(N,N-diethylamino)-diazen-1-ium-1,2-diolate (diethylamine NONOate or “DEA/NO”) and derivatives thereof). Still other vasoactive agents useful in conjunction with the topical administration of nitroglycerin herein include smooth muscle relaxants, leukotriene inhibitors, calcium channel blockers, β2-adrenergic agonists, angiotensin-converting enzyme (“ACE”) inhibitors, angiotensin II receptor antagonists, and phosphodiesterase inhibitors.

Still other suitable vasoactive agents include, but are not limited to: nitrates and like compounds other than nitroglycerin, e.g., isosorbide dinitrate, erythrityl tetranitrate, amyl nitrate, molsidomine, linsidomine chlorhydrate (“SIN-1”), S-nitroso-N-acetyl-d,1-penicillamine (“SNAP”) and S-nitroso-N-glutathione (“SNO-GLU”); long and short acting α-blockers such as phenoxybenzamine, dibenamine, doxazosin, terazosin, phentolamine, tolazoline, prazosin, trimazosin, alfuzosin, tamsulosin and indoramin; ergot alkaloids such as ergotamine and ergotamine analogs, e.g., acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride and terguride; antihypertensive agents such as diazoxide, hydralazine and minoxidil; nimodepine; pinacidil; cyclandelate; dipyridamole; isoxsuprine; chlorpromazine; haloperidol; yohimbine; and trazodone.

Still other secondary active agents for coadministration with the nitroglycerin are phosphodiesterase inhibitors, such as those described in U.S. Pat. Nos. 6,037,346, 6,127,363 and 6,156,753 to Place et al., and in U.S. Patent Application Publication 2002/0037828 to Wilson et al., and pending U.S. Ser. No. 09/467,094, filed Dec. 10, 1999, all of which are assigned to VIVUS, Inc. (Mountain View, Calif.). Suitable phosphodiesterase inhibitors include, but are not limited to, inhibitors of the type III phosphodiesterases (cAMP-specific-cGMP inhibitable form), the type IV phosphodiesterases (high affinity-high specificity cAMP form) and the type V phosphodiesterases (the cGMP specific form). Examples of type III phosphodiesterase inhibitors include, but are not limited to, bipyridines such as milrinone and amrinone, imidazolones such as piroximone and enoximone, dihydropyridazinones such as imazodan, 5-methylimazodan, indolidan and ICI118233, quinolinone compounds such as cilostamide, cilostazol and vesnarinone, and other compounds such as bemoradan, anergrelide, siguazodan, trequensin, pimobendan, SKF-94120 (5-(4-acetoamidophenyl)pyrazin-2-(1H)-one), SKF-95654, lixazinone and isomazole. Examples of type IV phosphodiesterase inhibitors include, but are not limited to, rolipram and rolipram derivatives such as R020-1724 (4-(3-butyloxy-4-methoxyphenyl)-imidazolidinone), nitraquazone and nitraquazone derivatives such as CP-77059 (1-(carbomethoxyphenyl)-3-benzyl-pyrido[2,3d]pyrimidine-2,4(1H,3H)dione) and RS-25344 (1-(3-nitrophenyl)-3-(4-pyridylmethyl)-1,2,3,4-tetrahydropyrido(2,3-d)pyrimidine-2,4-dione)), xanthine derivatives such as denbufylline and IC163197, and miscellaneous other compounds such as EMD54622 (5-[1-(3,4-dimethoxybenzoyl)-4,4-dimethyl-1,2,3,4-tetrahydrochinolin-6-yl]-6-methyl-3,4-dihydro-1,3,4-thiadizin-2-one), LAS-31025 (1-propyl-3-(4-chlorophenyl)-xanthine) and etazolate. Examples of type V phosphodiesterase inhibitors include, but are not limited to, zaprinast, MY5445 (1-(3-chloroanilino)-4-phenylphthalazine), dipyridamole, and sildenafil. Other suitable type V phosphodiesterase inhibitors are disclosed in PCT Publication Nos. WO 94/28902 and WO 96/16644.

Other secondary active agents herein are inhibitors of rho kinase, an enzyme belonging to the rhoA/rho associated kinase pathway, which regulates the state of phosphorylation of myosin phosphatase, in turn leading to the control of smooth muscle contraction. One example of a suitable rho kinase inhibitor has the structural formula

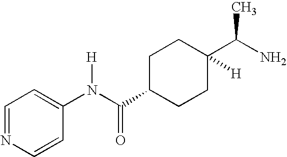

and is identified as Y-27632. Other suitable rho kinase inhibitors are disclosed, for example, in U.S. Pat. No. 6,218,410 to Uehata et al.

Additional secondary agents useful herein are peptide analogs of α-melanocyte-stimulating hormone (α-MSH), also referred to as “melanocortin peptides.” Such peptides are known in the art, and preferred ones are cyclic. Particularly preferred melanocortin peptides are described in U.S. Pat. No. 6,051,555 to Hadley and WO 01/00224 to Blood et al.

Suitable endothelin antagonists useful as secondary agents herein are antagonists of any or all of the three isoforms of endothelin, i.e., ET-1, ET-2, and ET-3, and are exemplified by: phenoxyphenylacetic acids and derivatives thereof, such as N-(4-isopropylbenzene-sulfonyl)-α-(4-carboxy-2-n-propylphenoxy)-3,4-methylenedioxyphenyl acetamide dipotassium salt, 2-[(2,6-dipropyl-4-hydroxymethyl)-phenoxy]-2-(4-phenoxyphenyl)-acetic acid, 2-[(2,6-dipropyl-4-hydroxymethyl)phenoxy]-2-(4-phenylphenyl)acetic acid, 2-[(2,6-dipropyl-4-hydroxymethyl)phenoxy]-2-(3-carboxyphenyl)-acetic acid, 2-[(2,6-dipropyl-4-hydroxymethyl)phenoxy]-2-(3,4-ethylenedioxyphenyl)acetic acid, 2-[(2,6-dipropyl-4-hydroxymethyl)phenoxy]-2-(3,4,5-trimethoxyphenyl)acetic acid, 2-[(2,6-dipropyl-4-hydroxymethyl)phenoxy]-2-(3,4-methylenedioxyphenyl)acetic acid, N-(4-dimethylaminobenzenesulfonyl)-2-(4-methoxycarbonyl-2-propylphenoxy)-2-(3,4-methylenedioxyphenyl) acetamide, N-(2-methylbenzenesulfonyl)-2-(4-methoxycarbonyl-2-propylphenoxy)-2-(3,4-methylenedioxyphenyl)acetamide, N-(2-methoxycarbonyl-benzenesulfonyl)-2-(4-methoxy-carbonyl-2-propylphenoxy)-2-(3,4-methylenedioxy-phenyl)acetamide, N-(2-chlorobenzene-sulfonyl)-2-(4-methoxycarbonyl-2-propyl-phenoxy)-2-(3,4-methylenedioxyphenyl)acetamide, and others, as described in U.S. Pat. No. 5,565,485 to Bagley, et al.; and certain isooxazoles, oxazoles, thiazoles, isothiazoles and imidazoles, as described, for example, in U.S. Pat. No. 6,136,828 to Elliott. Numerous other endothelin antagonists may be used as secondary agents herein, and will be known to those of ordinary skill in the art and/or are described in the pertinent patents, literature and texts.

Peptidyl drugs suitable as secondary active agents include, without limitation, activin, amylin, angiotensin, atrial natriuretic peptide (ANP), calcitonin, calcitonin gene-related peptide, calcitonin N-terminal flanking peptide, ciliary neurotrophic factor (CNTF), corticotropin (adrenocorticotropin hormone, ACTH), corticotropin-releasing factor (CRF or CRH), epidermal growth factor (EGF), follicle-stimulating hormone (FSH), gastrin, gastrin inhibitory peptide (GIP), gastrin-releasing peptide, gonadotropin-releasing factor (GnRF or GNRH), growth hormone releasing factor (GRF, GRH), human chorionic gonadotropin (hCH), inhibin A, inhibin B, insulin, luteinizing hormone (LH), luteinizing hormone-releasing hormone (LHRH), α-melanocyte-stimulating hormone, β-melanocyte-stimulating hormone, γ-melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, and vasopressin. Other peptidyl drugs are the cytokines, e.g., colony stimulating factor 4, heparin binding neurotrophic factor (HBNF), interferon-α, interferon α-2a, interferon α-2b, interferon α-n3, interferon-β, etc., interleukin-1, interleukin-2, interleukin-3, interleukin-4, interleukin-5, interleukin-6, etc., tumor necrosis factor, tumor necrosis factor-α, granuloycte colony-stimulating factor (G-CSF), granulocyte-macrophage colony-stimulating factor (GM-CSF), macrophage colony-stimulating factor, midkine (MD), and thymopoietin.

Selective androgen receptor modulators (SARMs) include LGD2226 and/or LGD1331, both available from Ligand Pharmaceuticals (San Diego, Calif.), and Casodex®, all of which are suitable secondary agents herein. See Negro-Villar et al. (1999) J. Clin. Endocrinol. & Metabol. 84(10): 3459-62. Other SARMS include, for example, cyproterone acetate, hydroxyflutamide, bicalutamide, spironolactone, 4-(trifluoromethyl)-2(1H)-pyrrolidone[3,2-g]quinolinone derivatives, 1,2-dihydropyridono[5,6-g]quinoline derivatives and piperidino[3,2-g]quinolinone derivatives.

Selective estrogen receptor modulators (SERMs) are compounds that produce tissue specific effects that can be agonistic or antagonistic to the effects of estrogen. SERMs include, without limitation:

benzothiophenes, including, but not limited to: raloxifene (Evista®, [6-hydroxy-3-[4-[2-(1-piperidinyl)ethoxy]phenoxy]-2-(4-hydroxyphenyl)] benzo[b]thiophene hydrochloride), and derivatives thereof, including —S—, —NH—, —NCH₃—, —SO₂— and —CH₂— substituted raloxifene, as described in Schmid et al. (1999) Bioorg. & Med. Chem. Lett. 9:523-528; trans-2,3-dihydroraloxifene; derivatives as disclosed in Grese, et al., J. Med. Chem. (1997) Vol. 40, pp. 146-167, such as 4′ halo raloxifene and 2-(alkyl, cycloalkyl or naphthyl) raloxifene; benzothiophenes as disclosed in U.S. Pat. No. 5,962,475 to Schmid, et al., such as 6-methoxy-2-(4-methoxyphenyl)-3-(4-nitrobenzoyl)-benzo[b]thiophene; arzoxifene (also known as Arzox or LY353381 (Lilly)), 2-(4-methoxyphenyl)-3-(4-(2-(1-piperidinyl)ethoxy)phenoxybenzo(b) thiophene-6-ol); LY 117018 (6-hydroxy-2-(4-hydroxyphenyl) benzo(b)thien-3-yl)(4-(2-(1-pyrrolidinyl)ethoxy)phenyl)-methanone), and bazedoxifen (TSE-424 (Ligand));

triphenylethylenes, including, but not limited to: idoxifene, 1-[2-[4-(1E)-1-(4-Iodophenyl)-2-phenyl-1-butenyl]phenoxy]ethyl] pyrrolidine; droloxifene, 3-[(1E)-1-[4-[2-(Dimethylamino)ethoxy]phenyl]-2-phenyl-1-butenyl]phenol; tamoxifen, (Z)-2-[4-(1,2-Diphenyl-1-butenyl)phenoxy]-N,N-dimethylethanamine; toremifene, 2-[4-[(1Z)-4-Chloro-1,2-diphenyl-1-butenyl)phenoxy]-N,N-dimethylethanamine; clomiphene, 2-[4-(2-Chloro-1,2-diphenylethenyl)phenoxy]-N,N-diethylethanamine; meproxifene (4-(1-(4-(2-(dimethylamino)ethoxy)phenyl)-2-(4-(1-methylethyl)phenyl)-1-butenyl)-phenol, or TAT-59 (Taiho)); trioxifene; zindoxifene; lasofoxifene; nafoxidine; halogenated triphenylethylene derivatives as disclosed in U.S. Patent Application Publication No. 2002/0013297 to Kaltenbach III et al., such as 3-[4-[1-(4-fluorophenyl)-2-phenyl-but-1-enyl]phenyl}acrylic acid, and 3-[4-(1,2-diphenyl-but-1-enyl)-phenyl]-acrylic acid;

substituted naphthalenes and isoquinolines, including, for example: cis-6-phenyl-5-(4-(2-pyrrolidin-1-yl-ethoxy)phenyl)-5,6,7,8-tetrahydronaphthalene-2-ol; cis-6-(4-fluorophenyl)-5-[4-(2-piperidin-1-yl-ethoxy)-phenyl]-5,6,7,8-tetrahydronaphthalene-2-ol; cis-1-[6′-pyrrolidinoethoxy-3′-pyridyl]-2-phenyl-6-hydroxy-1,2,3,4-tetrahydronaphthalene; cis-6-(4′-hydroxyphenyl)-5-[4-(2-piperidin-1-yl-ethoxy)-phenyl]-5,6,7,8-tetrahydronaphthalen-2-ol, 6-(4-hydroxyphenyl)-5-[4-(2-piperidin-1-yl-ethoxy)-benzyl]-naphthalen-2-ol; 1-(4′-pyrrolidino-ethoxyphenyl)-2-(4″-fluoroophenyl)-6-hydroxy-1,2,3,4-tetrahydroisoquinoline; 1-(4′-pyrrolidino-ethoxyphenyl)-2-phenyl-6-hydroxy-1,2,3,4 tetrahydroisoquinoline; and other compounds disclosed in U.S. Pat. No. 5,916,916 to Hauser, et al., U.S. Pat. No. 5,552,412 to Cameron et al., and in EP 1004306 A2;

benzopyrans, including but not limited to: 2,3 diaryl-2H-1-benzopyrans, as described by Sharma et al. (1990) J. Med. Chem. 33:3216, having tertiary aminoethoxy substituents such as piperidinoethoxy, pyrrolidino and dimethylamino at the para position of the 2-phenyl, and alkyl substituents at the 4 position of the pyran ring; 4-fluoroalkyl-2H-benzopyrans, as disclosed in WO 01/68634;

steroids and estrols including: tibolone; diethylstilbestrol; moxestrol; N-butyl-3,17-dihydroxy-N-methyl-estra-1,3,5(10)-triene-7-undecanamide (ICI 164,384); fulvestrant (Faslodex®, ICI 182,780); 19-nor-progesterone derivatives, and 19-nor-testosterone derivatives;

substituted coumarin and chromen-2-one compounds, including but not limited to: compounds disclosed in WO 01/49673, preferably 3-phenyl-4-[4-(2-(piperadin-1-yl))ethoxy]-benzyl-7-hydroxycoumarin, 3-(4-chlorophenyl)-4-[4-(2-(piperadin-1-yl))ethoxy]-phenyl-7-hydroxycoumarin as well as derivatives wherein the piperidino group is substituted with diethylamino; chroman; centchroman; and levormeloxifene;

phytoestrogens, such as genistein;

diphenol compounds having estrogenic activity, such as hexestrol;

and salts, esters and derivatives and combinations of any of the foregoing.

Suitable neuropeptides useful as secondary active agents include bradykinin, kallidin, des-Arg⁹-bradykinin, des-Arg¹⁰-kallidin, des-Arg⁹-[Leu⁸]-bradykinin, [D-Phe⁷]-bradykinin, HOE 140, neuropeptide Y, calcitonin gene-related peptide (cGRP), enkephalins and related opioid peptides such as Met⁵-enkephalin, Leu⁵-enkephalin, α-, β- and γ-endorphin, α- and β- neo-endorphin, and dynorphin, as well as the neurotransmitters GABA (γ-aminobutyric acid), glycine, glutamate, acetylcholine, dopamine, epinephrine, 5-hydroxytryptamine, substance P, serotonin, and catecholamines.

One or more amino acids may also be included in the present formulations as a secondary active agent. As used herein, the term “amino acid” includes the conventional amino acids, e.g., phenylalanine, leucine, isoleucine, methionine, valine, serine, proline, threonine, alanine, tyrosine, histidine, glutamine, asparagine, lysine, aspartic acid, glutamic acid, cysteine, tryptophan, arginine, and glycine, with arginine being particularly preferred. In addition, the term “amino acid” will also include amino acid derivatives, e.g., 1-naphthylalanine, 2-naphthylalanine, 3-pyridylalanine, 4-hydroxyproline, O-phosphoserine, N-acetylserine, N-formylmethionine, 3-methylhistidine, 5-hydroxylysine, and nor-leucine, in addition to stereoisomers (e.g., D-amino acids) of the twenty conventional amino acids. Combinations of any of the foregoing are contemplated as well. Preferred amino acids are the neuroactive amino acids γ-aminobutyric acid (GABA), glycine, α-alanine, taurine, and glutamate.

Suitable serotonin agonists include, but are not limited to, 2-methyl serotonin, buspirone, ipsaperone, tiaspirone, gepirone, ergot alkaloids, 8-hydroxy-(2-N,N-dipropyl-amino)-tetraline, 1-(4-bromo-2,5-dimethoxyphenyl)-2-aminopropane, cisapride, sumatriptan, m-chlorophenylpiperazine, trazodone, zacopride, mezacopride, and combinations thereof. Suitable serotonin antagonists include, for example, ondansetron, granisetron, metoclopramide, tropisetron, dolasetron, palonosetron, trimethobenzamide, methysergide, risperidone, ketanserin, ritanserin, clozapine, amitriptyline, MDL 100,907 (R(+)-α-(2,3-dimethoxyphenyl)-1-[2-(4-fluorophenyl)ethyl]-4-piperidine-methanol) (Marion Merrell Dow), azatadine, cyproheptadine, fenclonine, chlorpromazine, mianserin and combinations thereof.

Representative ergot alkaloids include ergotamine and ergotamine analogs, e.g., acetergamine, brazergoline, bromerguride, cianergoline, delorgotrile, dihydroergotamine, disulergine, ergonovine, ergonovine maleate, ergotamine tartrate, etisulergine, lergotrile, lysergide, mesulergine, metergoline, metergotamine, nicergoline, pergolide, propisergide, proterguride and terguride.

Calcium channel blockers that are suitable for use as secondary agents herein include, without limitation, amlodipine, felodipine, isradipine, nicardipine, nifedipine, nimodipine, nisoldipine, nitrendipine, bepridil, diltiazem, verapamil, and combinations thereof.

Potassium channel openers include, but are not limited to, pinacidil, diazoxide, cromakalim, nicorandil, minoxidil, (N-cyano-N′-(1,1-dimethylpropyl)-N″-3-pyridylguanidine (P-1075), and N-cyano-N′-(2-nitroxyethyl)-3-pridinecarboximidamide monomethanesulfonate (KRN 2391). Potassium channel blockers include tedisamil, agitoxin-2, apamin, BDS-I, BDS-II, charybdotoxin, α-dendrotoxin, β-dendrotoxin, γ-dendrotoxin, 6-dendrotoxin, dendrotoxin-I, dendrotoxin-K, E-403 1, iberiotoxin, kaliotoxin, MCD-peptide, margatoxin, noxiustoxin, paxilline, penitrem A, stichodactyla, tertiapin, tityustoxin K alpha, verruculogen, and combinations thereof. Although all of the active agents are available commercially, most of the listed potassium channel blockers are available from Alomone Labs (Jerusalem, Israel).

Suitable dopamine agonists include, for example, levodopa, bromocriptine, pergolide, apomorphine, piribedil, pramipexole, ropinirole, and combinations thereof. Dopamine antagonists include, without limitation, spiroperidol, benperidol, trifluperidol, pimozide, fluphenazine, droperidol, haloperidol, thiothixene, trifluperazine, moperone, prochlorperazine, molindone, thioridazine, clozapine, chlorpromazine, promazine, sulpiride, clebopride, chlorpromazine, spiperone, flupenthixol, and combinations thereof.

Steroidal agents that may be administered as secondary active agents include progestins, estrogens and androgens.

Suitable estrogens include synthetic and natural estrogens such as: estradiol (i.e., 1,3,5-estratriene-3,17β-diol, or “17β-estradiol”) and its esters, including estradiol benzoate, valerate, cypionate, heptanoate, decanoate, acetate and diacetate; 17α-estradiol; ethinylestradiol (i.e., 17α-ethynylestradiol) and esters and ethers thereof, including ethinylestradiol 3-acetate and ethinylestradiol 3-benzoate; estriol and estriol succinate; polyestrol phosphate; estrone and its esters and derivatives, including estrone acetate, estrone sulfate, and piperazine estrone sulfate; quinestrol; mestranol; and conjugated equine estrogens. Preferred estrogens are 17β-estradiol, 17α-estradiol, ethinylestradiol and mestranol.

Suitable progestins include acetoxypregnenolone, allylestrenol, anagestone acetate, chlormadinone acetate, cyproterone, cyproterone acetate, desogestrel, dihydrogesterone, dimethisterone, ethisterone (17α-ethinyltestosterone), ethynodiol diacetate, flurogestone acetate, gestadene, hydroxyprogesterone, hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, and progesterone. Preferred progestins are progestins such as cyproterone, cyproterone acetate, hydroxyprogesterone, levonorgestrel, medroxyprogesterone acetate, norethindrone and norgestrel.

It is generally desirable to co-administer a progestin along with an estrogen so that the estrogen is not “unopposed.” As is well known in the art, estrogen-based therapies are known to increase the risk of endometrial hyperplasia and cancer, as well as the risk of breast cancer, in treated individuals. Co-administration of an estrogen with a progestin has been found to decrease the aforementioned risks.

Suitable androgens include, but are not limited to the naturally occurring androgens and derivatives thereof, including androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, ethylestrenol, oxandrolone, nandrolone phenpropionate, nandrolone decanoate, nandrolone furylpropionate, nandrolone cyclohexane-propionate, nandrolone benzoate, nandrolone cyclohexanecarboxylate, stanozolol, dromostanolone, dromostanolone propionate, testosterone, dehydroepiandrosterone (DHEA; also termed “prasterone”), sodium dehydroepiandrosterone sulfate, and 4-dihydrotestosterone (DHT; also referred to as “stanolone” and 5α-dihydrotestosterone); pharmaceutically acceptable esters of testosterone and 4-dihydrotestosterone, typically esters formed from the hydroxyl group present at the C-17 position, including, but not limited to, the enanthate, propionate, cypionate, phenylacetate, acetate, isobutyrate, buciclate, heptanoate, decanoate, pentadecanoate, undecanoate, pelargonate, tridecanoate, palmitate, caprate, isocaprate, α-methylcaprate, β-methylcaprate, laurate, α-methylpelargonate, β-methylpelargonate, β,β-dimethylpelargonate, β-(p-methyl-cyclohexyl)propionate, β-(p-ethylcyclohexyl)-propionate, β-(cycloheptyl)-propionate, α-methyl-cyclohexyl-propionate, β-methyl-β-cyclohexyl-propionate, cyclododecyl-carboxylate, adamantine-1′-carboxylate, adamant-1′-yl-acetate, methyl-α-cyclohexyl propionate, and α-(bicyclo-[2,2,2-oct-1′-yl)-propionate esters, as well as the alkyl-substituted, preferably C₄-C₆ alkyl-substituted cyclic esters, such as the 3-n-hexylcyclo-butanecarboxylate, 3-n-butylcyclopentanecarboxylate, 4-n-butylcyclohexanecarboxylate, 4-n-pentylcyclohexanecarboxylate and n-hexylcyclohexanecarboxylate esters (and other such esters disclosed in U.S. Pat. No. 4,948,790 to Archer et al.); and pharmaceutically acceptable derivatives of testosterone such as methyl testosterone, testolactone, oxymetholone, fluoxymesterone, and the like, and combinations of any of the foregoing.

Any of the secondary active agents may be administered in the form of a salt, ester, amide, inclusion complex, prodrug, metabolite, analog, or other derivative, and such derivatives may be prepared using standard procedures known to those skilled in the art of synthetic organic chemistry and described, for example, by J. March, Advanced Organic Chemistry: Reactions, Mechanisms and Structure, 4^{th} Ed. (New York: Wiley-Interscience, 1992). For example, acid addition salts are prepared from an active agent in the form of a free base (typically wherein the neutral form of the drug has a neutral —NH₂ group) using conventional means, involving reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of acid moieties that may be present on a drug (e.g., the carboxylic acid functionality of prostanoic acid) are prepared in a similar manner using a pharmaceutically acceptable base such as sodium hydroxide, potassium hydroxide, ammonium hydroxide, calcium hydroxide, trimethylamine, or the like. Preparation of esters involves transformation of a carboxylic acid group via a conventional esterification reaction involving nucleophilic attack of an RO⁻ moiety at the carbonyl carbon. Esterification may also be carried out by reaction of a hydroxyl group with an esterification reagent such as an acid chloride. Esters can be reconverted to the free acids, if desired, by using conventional hydrolysis procedures. Amides may be prepared from esters, using suitable amine reactants, or they may be prepared from an anhydride or an acid chloride by reaction with ammonia or a lower alkyl amine.

Prodrugs and active metabolites may also be prepared using techniques known to those skilled in the art or described in the pertinent literature. Prodrugs are typically prepared by covalent attachment of a moiety that results in a compound that is therapeutically inactive until modified by an individual's metabolic system.

Inclusion complexes are complexes formed by interaction of macrocyclic compounds containing an intramolecular cavity of molecular dimensions with the smaller, pharmacologically active agent. Preferred inclusion complexes are formed from α-, β- and γ-cyclodextrins, or from clathrates, in which the “host” molecules form a crystal lattice containing spaces in which “guest” molecules (e.g., the nitroglycerin and any secondary active agents) will fit. See, e.g., Hagan, Clathrate Inclusion Compounds (New York: Reinhold, 1962). Cyclodextrin complexes of the prostaglandin may be used in order to increase the stability and efficacy of the formulation. Cyclodextrin complexes may be prepared by adding the proper stoichiometric ratio of the prostaglandin to α-, β- or γ-cyclodextrin in an aqueous solvent and then either using as is or lyophilizing to provide a solid clathrate. These complexes are described in Yamamura et al. (1985) J. Chromatogr. 331(2):383-388, Hirayama et al. (1984) Chem. Pharm. Bull. (Tokyo) 32(10):4237-4240, Uekama et al. (1984) J. Pharm. Sci. 73(3):382-384, and Yamamura et al. (1984) J. Chromatogr. 303(1):165-172. Other methods known in the art for forming inclusion complexes may also be used. Prodrugs, active metabolites and other derivatives can be prepared using techniques known to those of ordinary skill in the art and/or using syntheses described in the pertinent texts and literature.

IV. Dosage and Administration

The amount and/or concentration of the active agent(s) in any of the aforementioned dosage forms and compositions can vary a great deal, and will depend on a variety of factors, including the type of dosage form, the corresponding mode of administration, the intended release profile, the nature and activity of any secondary active agents, the age and general condition of the individual being treated, the severity of the individual's condition, and other factors known to the prescribing physician.

Preferred dosage forms contain a unit dose of active agent, i.e., a single therapeutically effective dose. For solutions, gels, creams, lotions, ointments, pastes, foams, etc., a “unit dose” requires an active agent concentration that provides a unit dose in a specified quantity of the formulation to be applied. The unit dose of any particular active agent will depend, of course, on a number of factors. Typically, a “unit dosage” of nitroglycerin that is administered in a single application is in the range of about 0.05 mg to about 150 mg, typically about 0.1 mg to about 30 mg, preferably about 0.2 mg to about 20 mg, and most preferably about 0.2 mg to about 10 mg. The formulation may be administered on an as-needed basis, or on an ongoing basis, for example once, twice or three times daily. Preferably, the formulation is administered on an as-needed basis. The optimal dosage and course of therapy for a given patient can be readily ascertained by those skilled in the art using conventional course of therapy determination tests and taking into account the information provided herein.

The amount of a particular secondary active agent administered to a given individual will, of course, be dependent on a number of factors as well, including the specific active agent, composition or dosage form, the selected mode of administration, and the like.

In a preferred embodiment, drug administration is on an as-needed basis, and does not involve chronic drug administration. That is, an immediate release dosage form may be used to administer the drug, such that substantially all of the drug (i.e., greater than 80% by weight, preferably greater than 90%) is released from the composition or dosage form within about 4 hours, preferably within about 2 hours, most preferably within about 1 hour, following administration. With a sustained release dosage form, a single dose can provide therapeutic efficacy over an extended time period in the range of about 4 to 48 hours, typically in the range of about 4 to 24 hours, depending on the formulation. The release period may be varied by the selection and relative quantity of particular sustained release polymers. If necessary, drug administration may be carried out within the context of an ongoing dosage regimen, i.e., on a weekly basis, twice weekly, daily, twice daily, etc.

V. Packaged Kits

In another embodiment, a packaged kit is provided that contains the pharmaceutical formulation to be administered, i.e., a pharmaceutical formulation containing a sufficient quantity of nitroglycerin for the treatment of dyspareunia, a container, preferably sealed, for housing the formulation during storage and prior to use, and instructions for carrying out drug administration in a manner effective to treat dyspareunia. The instructions will typically be written instructions on a package insert and/or on a label. The formulation may be any suitable formulation as described herein. For example, the formulation may be an ampoule, capsule, or other dispenser containing a nitroglycerin solution optionally further containing a secondary active agent (e.g., a single use ampoule containing a unit dosage of each active agent), or it may be a tube containing a nitroglycerin cream, ointment, lotion, etc. (again, the formulation may contain one or more secondary active agents, and the tube may be a single use dispenser containing a unit dosage of each active agent).

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents, patent applications, patent publications and non-patent literature references mentioned herein are incorporated by reference in their entireties.

### EXPERIMENTAL

The practice of the present invention will employ, unless otherwise indicated, conventional techniques of pharmaceutical formulation, medicinal chemistry, and the like, which are within the skill of the art. Such techniques are explained fully in the literature. Preparation of various types of pharmaceutical formulations are described, for example, in Remington: The Science and Practice of Pharmacy, 20^{th} edition (Lippincott Williams & Wilkins, 2000) and Ansel et al., Pharmaceutical Dosage Forms and Drug Delivery Systems, 6^{th} Ed. (Media, PA: Williams & Wilkins, 1995).

In the following examples, efforts have been made to ensure accuracy with respect to numbers used but some experimental error and deviation should be accounted for. Unless indicated otherwise, temperature is in degrees C. and pressure is at or near atmospheric. All components were obtained commercially unless otherwise indicated.

### Example 1

This example describes the preparation and use of a nitroglycerin solution for application to the vulvar region of a female individual suffering from dyspareunia. The solution can be prepared by mixing the following components:

| Component | % w/w | wt. per 1.0 g formulation |
|---|---|---|
| Nitroglycerin, USP | 0.3 | 3.0 mg |
| Isopropyl myristate, NF | 32.0 | 320 mg |
| Cyclomethicone DC344, NF | 60.0 | 600 mg |
| Ethanol, 200 proof, USP | 7.7 | 77 mg |
| Total | 100 | 1000 mg |

The bulk solutions can be filled into plastic unit dose containers, e.g., ampoules, capsules, or other dispensers, at a fill volume of 1.0 mL, resulting in a unit dose of nitroglycerin of approximately 3.0 mg.

### Example 2

The procedure of Example 1 is repeated except that the following components are used:

| Component | % w/w | wt. per 1.0 g formulation |
|---|---|---|
| Nitroglycerin, USP | 0.1 | 1.0 mg |
| Isopropyl myristate, NF | 32.0 | 320 mg |
| Cyclomethicone DC344, NF | 60.0 | 600 mg |
| Ethanol, 200 proof, USP | 7.9 | 79 mg |
| Total | 100 | 1000 mg |

### Example 3

The procedure of Example 1 is repeated except that the following components are used:

| Component | % w/w | wt. per 1.0 g formulation |
|---|---|---|
| Nitroglycerin, USP | 1.0 | 10 mg |
| Isopropyl myristate, NF | 32.0 | 320 mg |
| Cyclomethicone DC344, NF | 60.0 | 600 mg |
| Ethanol, 200 proof, USP | 7.0 | 70 mg |
| Total | 100 | 1000 mg |

### Example 4

The procedure of Example 1 is repeated except that the following components are used:

| Component | % w/w | wt. per 1.0 g formulation |
|---|---|---|
| Nitroglycerin, USP | 0.2 | 2 mg |
| Alprostadil (PGE₁), USP | 0.1 | 1 mg |
| Isopropyl myristate, NF | 32.0 | 320 mg |
| Cyclomethicone DC344, NF | 60.0 | 600 mg |
| Ethanol, 200 proof, USP | 7.7 | 77 mg |
| Total | 100 | 1000 mg |

### Example 5

An ointment formulation for topical administration of nitroglycerin in the treatment of dyspareunia is prepared with the following components:

| Component | % w/w | wt. per 1.0 g formulation |
|---|---|---|
| Nitroglycerin, USP | 0.3 | 3.0 mg |
| Anhydrous lanolin | 24.0 | 240 mg |
| Mineral oil | 26.0 | 260 mg |
| White petrolatum | 49.7 | 497 mg |
| Total | 100 | 1000 mg |

Mixing is conducted with tile and spatula until a homogeneous ointment mixture is obtained having the nitroglycerin uniformly dispersed throughout the formulation.

### Example 6

An ointment formulation for topical administration of nitroglycerin and alprostadil in the treatment of dyspareunia is prepared using the procedures of Example 5, with the following components:

| Component | % w/w | wt. per 1.0 g formulation |
|---|---|---|
| Nitroglycerin, USP | 0.3 | 3.0 mg |
| Alprostadil, USP | 0.1 | 1.0 mg |
| Anhydrous lanolin | 24.0 | 240 mg |
| Mineral oil | 26.0 | 260 mg |
| White petrolatum | 49.6 | 496 mg |
| Total | 100 | 1000 mg |

### Example 7

Suppositories suitable for either vaginal or vulvar administration of nitroglycerin are prepared. Initially, a composition is prepared by mixing 3.0 mg nitroglycerin with 0.97 g polyethylene glycol, molecular weight (M_{w}) approximately 4000, and heating the mixture to a temperature just high enough to produce a drug-polymer melt. The mixture can then be poured into a mold suitable to provide a suppository, and allowed to cool. The suppository so provided is a unit dosage form suitable for vaginal or vulvar administration.

### EXAMPLE 8

Individuals are assessed and pre-screened to assemble an experimental group of subjects suffering from dyspareunia. The compositions prepared in Examples 1-8, formulated with nitroglycerin and optionally alprostadil as well, are each assessed in the experimental subjects for their ability to reduce or eliminate the symptoms associated with dyspareunia, including pain and discomfort during sexual intercourse and post-coital vaginal burning, pelvic aching, urinary discomfort, and lower abdominal distress. A unit dose of active agent(s) is administered to each subject once daily. Dyspareunia is negatively correlated with vaginal blood flow rates, wherein increased blood flow to the vagina correlates with increased lubrication and decreased frequency and severity of dyspareunia (Sarrel, P. M. (1990) Obstet. Gynaecol. 75:26S-32S). Accordingly, following administration of a unit dose of the active agent(s), changes in blood flow or vaginal fluid production after application of the vasodilating formulations are determined using known methods. Increase in vaginal epithelial blood flow may be determined using indirect methods such as photoplethysmography (Levin (1980) Clinics in Obstet. Gynaecol. 7:213-252), heated oxygen electrode (Wagner et al. (1978), “Vaginal Fluid” in The Human Vagina, Evans et al. (eds.), Amsterdam: Elsevier/North Holland Biomedical Press, pp. 121-137), and direct clearance of radioactive Xenon (Wagner et al. (1980) Obstet. Gynaecol. 56:621-624). Changes in vulvar blood flow are monitored using laser Doppler velocimetry (Sarrel, P. M. (1990) Obstet. Gynaecol. 75:26S-32S).

The compositions of Examples 1-8, when assessed using such methods, are found to substantially reduce or eliminate the manifestations of dyspareunia.

We claim:

1. A method for treating dyspareunia in a female individual, comprising administering to the individual's vulvar region or vagina a pharmaceutical formulation that comprises nitroglycerin in an amount effective to alleviate or eliminate the symptoms of dyspareunia, in combination with a pharmaceutically acceptable carrier.

2. The method of claim 1, wherein the pharmaceutical formulation is a topical formulation, and is administered to the patient's vulvar region.

3. The method of claim 2, wherein the pharmaceutical formulation is suitable for vaginal administration and is administered vaginally.

4. The method of claim 1, wherein the pharmaceutical formulation comprises a unit dosage form.

5. The method of claim 1, wherein the pharmaceutical formulation is selected from the group consisting of solutions, gels, creams, ointments, suspensions, pastes, foams, and suppositories.

6. The method of claim 1, further comprising administering a therapeutically effective amount of at least one additional active agent.

7. The method of claim 6, wherein the at least one additional active agent is administered with the nitroglycerin.

8. The method of claim 7, wherein the additional active agent and the nitroglycerin are contained in the same formulation.

9. The method of claim 6, wherein the at least one additional active agent is administered prior to administration of the nitroglycerin.

10. The method of claim 6, wherein the at least one additional active agent is administered after administration of the nitroglycerin.

11. The method of claim 6, wherein the at least one additional active agent is a vasoactive agent.

12. The method of claim 11, wherein the vasoactive agent is selected from the group consisting of naturally occurring prostaglandins, synthetic prostaglandin derivatives, endothelian-derived relaxation factors, vasoactive intestinal polypeptide agonists, smooth muscle relaxants, leukotriene inhibitors, calcium channel blockers, phosphodiesterase inhibitors, nitrates, α-receptor blocking agents, ergotamine drugs, antihypertensive agents, pharmacologically acceptable salts, esters, prodrugs, active metabolites and inclusion complexes thereof, and combinations of any of the foregoing.

13. The method of claim 12, wherein the vasoactive agent is a naturally occurring prostaglandin or a hydrolyzable lower alkyl ester thereof.

14. The method of claim 13, wherein the naturally occurring prostaglandin is selected from the group consisting of PGE₀, PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF_{3α}, PGI₂, and combinations thereof.

15. The method of claim 14, wherein the naturally occurring prostaglandin is PGE₀ or a lower alkyl ester thereof.

16. The method of claim 14, wherein the naturally occurring prostaglandin is PGE₁ or a lower alkyl ester thereof.

17. The method of claim 11, wherein the vasoactive agent is a synthetic prostaglandin derivative or a hydrolyzable lower alkyl ester thereof.

18. The method of claim 17, wherein the synthetic prostaglandin derivative is selected from the group consisting of carboprost tromethamine, dinoprost tromethamine, gemeprost, metenoprost, sulprostone and tiaprost.

19. The method of claim 6, wherein the at least one additional active agent is a phosphodiesterase inhibitor.

20. The method of claim 6, wherein the at least one additional active agent is an androgen.

21. The method of claim 1, wherein the pharmaceutical formulation is contained within a delivery system selected to provide a predetermined agent release profile.

22. The method of claim 21, wherein the pharmaceutical formulation is an immediate release dosage form.

23. The method of claim 21, wherein the pharmaceutical formulation is a sustained release dosage form.

24. The method of claim 3, wherein the pharmaceutical formulation is contained within a vaginal ring, tampon, suppository, sponge, pillow, puff, or osmotic pump system.

25. A pharmaceutical formulation for treating dyspareunia, comprising a liquid or semi-solid formulation containing approximately 0.001 wt % to about 15.0 wt % nitroglycerin in a pharmaceutically acceptable carrier for vulvar and/or vaginal administration, wherein the carrier is effective to promote immediate drug release following vulvar and/or vaginal administration, and the total concentration Q of nitroglycerin in the formulation is greater than the solubility C_{S} of the nitroglycerin in the formulation.

26. The formulation of claim 25, wherein the formulation contains approximately 0.01 wt % to about 10.0 wt % nitroglycerin.

27. The formulation of claim 26, wherein the formulation contains approximately 0.1 wt % to about 5 wt % nitroglycerin.

28. The formulation of claim 27, wherein the formulation contains approximately 0.3 wt % to about 3.0 wt % nitroglycerin.

29. The formulation of claim 25, wherein Q is at least 50% greater than Cs.

30. The formulation of claim 25, wherein Q is at least 75% greater than Cs.

31. The formulation of claim 25, wherein the pharmaceutically acceptable carrier is hydrophobic.

32. The formulation of claim 31, wherein the pharmaceutically acceptable carrier is selected from the group consisting of fatty acids, fatty alcohols, bile acids, esters of fatty acids, fatty esters of alcohol, polyoxyalkylene fatty acid esters, polyoxyalkylene sorbitan fatty acid esters, and combinations thereof.

33. The formulation of claim 25, wherein a minimum of 80 wt % of the nitroglycerin in the formulation is released within 4 hours of administration.

34. The formulation of claim 33, wherein a minimum of 80 wt % of the nitroglycerin in the formulation is released within 2 hours of administration.

35. The formulation of claim 34, wherein a minimum of 80 wt % of the nitroglycerin in the formulation is released within 1 hour of administration.

36. The formulation of claim 25, further including at least one additional active agent.

37. The formulation of claim 36, wherein the at least one additional active agent is a vasoactive agent.

38. The formulation of claim 37, wherein the vasoactive agent is selected from the group consisting of naturally occurring prostaglandins, synthetic prostaglandin derivatives, endothelial-derived relaxation factors, vasoactive intestinal polypeptide agonists, smooth muscle relaxants, leukotriene inhibitors, calcium channel blockers, phosphodiesterase inhibitors, nitrates, α-receptor blocking agents, ergotamine drugs, antihypertensive agents, pharmacologically acceptable salts, esters, prodrugs, active metabolites and inclusion complexes thereof, and combinations of any of the foregoing.

39. The formulation of claim 38, wherein the vasoactive agent is a naturally occurring prostaglandin or a hydrolyzable lower alkyl ester thereof.

40. The formulation of claim 39, wherein the naturally occurring prostaglandin is selected from the group consisting of PGE₀, PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF_{3α}, PGI₂, and combinations thereof.

41. The formulation of claim 40, wherein the naturally occurring prostaglandin is PGEo or a lower alkyl ester thereof.

42. The formulation of claim 40, wherein the naturally occurring prostaglandin is PGE, or a lower alkyl ester thereof.

43. The formulation of claim 38, wherein the vasoactive agent is a synthetic prostaglandin derivative or a hydrolyzable lower alkyl ester thereof.

44. The formulation of claim 43, wherein the synthetic prostaglandin derivative is selected from the group consisting of carboprost tromethamine, dinoprost tromethamine, gemeprost, metenoprost, sulprostone and tiaprost.

45. The formulation of claim 36, wherein the at least one additional active agent is a phosphodiesterase inhibitor.

46. The formulation of claim 36, wherein the at least one additional active agent is an androgen.

47. The formulation of claim 46, wherein the androgen is selected from the group consisting of androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, ethylestrenol, oxandrolone, nandrolone phenpropionate, nandrolone decanoate, nandrolone furylpropionate, nandrolone cyclohexane-propionate, nandrolone benzoate, nandrolone cyclohexanecarboxylate, stanozolol, dromostanolone, dromostanolone propionate, testosterone, dehydroepiandrosterone, sodium dehydroepiandrosterone sulfate, 4-dihydrotestosterone, pharmaceutically acceptable esters thereof, alkyl substitutions thereof, derivatives thereof, and combinations of any of the foregoing.

48. The formulation of claim 47, wherein the androgen is testosterone, dehydroepiandrosterone or 4-dihydrotestosterone and pharmaceutically acceptable esters thereof.

49. The formulation of claim 47, wherein the pharmaceutically acceptable esters are selected from the group consisting of enanthate, propionate, cypionate, phenylacetate, acetate, isobutyrate, buciclate, heptanoate, decanoate, pentadecanoate, undecanoate, pelargonate, tridecanoate, palmitate, caprate, isocaprate, α-methylcaprate, β-methylcaprate, laurate, α-methylpelargonate, β-methylpelargonate, β,β-dimethylpelargonate, β-(p-methyl-cyclohexyl)propionate, α-(p-ethylcyclohexyl)-propionate, α-(cycloheptyl)-propionate, α-methyl-α-cyclohexyl-propionate, β-methyl-β-cyclohexyl-propionate, cyclododecyl-carboxylate, adamantine-1′-carboxylate, adamant-1′-yl-acetate, methyl-β-cyclohexyl propionate, β-(bicyclo-[2,2,2-oct-1′-yl)-propionate esters and alkyl-substituted cyclic esters.

50. The formulation of claim 49, wherein the alkyl-substituted cyclic esters are selected from the group consisting of 3-n-hexylcyclobutanecarboxylate, 3-n-butylcyclopentanecarboxylate, 4-n-butylcyclohexanecarboxylate, 4-n-pentylcyclohexanecarboxylate and n-hexylcyclohexanecarboxylate.

51. The formulation of claim 36, wherein the at least one additional active agent is a selective androgen receptor modulator.

52. The formulation of claim 51, wherein the selective androgen receptor modulator is selected from the group consisting of LGD-2226, LGD-1331, Casodex®, cyproterone acetate, hydroxyflutamide, bicalutamide, spironolactone, 4-(trifluoromethyl)-2(1H)-pyrrolidone[3,2-g]quinolinone, 1,2-dihydropyridono[5,6-g]quinoline and piperidino[3,2-g]quinolinone, derivatives thereof, salts and esters thereof and combinations thereof.

53. The formulation of claim 38, wherein the at least one additional active agent is an estrogen, a progestin or combinations thereof.

54. The formulation of claim 53, wherein the estrogen is 17β-estradiol, 17α-estradiol, ethinylestradiol, estriol, polyestrol phosphate, estrone, quinestrol, mestranol, conjugated equine estrogens, pharmaceutically acceptable esters thereof, derivatives thereof and combinations of any of the foregoing.

55. The formulation of claim 54, wherein the estrogen is 17β-estradiol, 17α-estradiol, ethinylestradiol, mestranol, pharmaceutically acceptable esters thereof, derivatives thereof and combinations of any of the foregoing.

56. The formulation of claim 53, wherein the progestin is selected from the group consisting of acetoxypregnenolone, allylestrenol, anagestone acetate, chlormadinone acetate, cyproterone, cyproterone acetate, desogestrel, dihydrogesterone, dimethisterone, ethisterone, ethynodiol diacetate, flurogestone acetate, gestadene, hydroxyprogesterone, hydroxymethylprogesterone, 3-ketodesogestrel, levonorgestrel, lynestrenol, medrogestone, medroxyprogesterone acetate, megestrol, melengestrol acetate, norethindrone, norethisterone, norethynodrel, norgestimate, norgestrel, norgestrienone, normethisterone, progesterone, pharmaceutically acceptable esters thereof, and combinations of any of the foregoing.

57. The formulation of claim 56, wherein the progestin is selected from the group consisting of cyproterone, cyproterone acetate, hydroxyprogesterone, levonorgestrel, medroxyprogesterone acetate, norethindrone, norgestrel, pharmaceutically acceptable esters thereof, and combinations of any of the foregoing.

58. The formulation of claim 36, wherein the at least one additional active agent is a selective estrogen receptor modulator.

59. The formulation of claim 58, wherein the selective estrogen receptor modulator is selected from the group consisting of benzothiophenes, triphenylethylenes, naphthalenes, isoquinolines, benzopyrans, steroids, coumarins, phytoestrogens, diphenols, and salts, esters and derivatives and combinations of any of the foregoing.

60. The formulation of claim 59, wherein the benzothiophene is selected from the group consisting of raloxifene, trans-2,3-dihydroraloxifene, 4′-halo raloxifene, 2-alkyl raloxifene, 2-cycloalkyl raloxifene, 2-naphthyl raloxifene, 6-methoxy-2-(4-methoxyphenyl)-3-(4-nitrobenzoyl)-benzo[b]thiophene, arzoxifene, 6-hydroxy-2-(4-hydroxyphenyl) benzo(b)thien-3-yl)(4-(2-(1-pyrrolidinyl)ethoxy)phenyl)-methanone), and bazedoxifen.

61. The formulation of claim 59, wherein the triphenylethylene is selected from the group consisting of idoxifene, droloxifene, tamoxifen, toremifene, clomiphene, meproxifene, trioxifene, zindoxifene, lasofoxifene, nafoxidine, and halogenated triphenylethylenes.

62. The formulation of claim 61, wherein the halogenated triphenylethylene is 3-[4-[1-(4-fluorophenyl)-2-phenyl-but-1-enyl]phenyl}acrylic acid or 3-[4-(1,2-diphenyl-but-1-enyl)-phenyl]-acrylic acid.

63. The formulation of claim 59, wherein the naphthalene is selected from the group consisting of cis-6-phenyl-5-(4-(2-pyrrolidin-1-yl-ethoxy)phenyl)-5,6,7,8-tetrahydronaphthalene-2-ol, cis-6-(4-fluorophenyl)-5-[4-(2-piperidin-1-yl-ethoxy)-phenyl]-5,6,7,8-tetrahydronaphthalene-2-ol, cis-1-[6′-pyrrolidinoethoxy-3′-pyridyl]-2-phenyl-6-hydroxy-1,2,3,4-tetrahydronaphthalene, cis-6-(4′-hydroxyphenyl)-5-[4-(2-piperidin-1-yl-ethoxy)-phenyl]-5,6,7,8-tetrahydronaphthalen-2-ol, and 6-(4-hydroxyphenyl)-5-[4-(2-piperidin-1-yl-ethoxy)-benzyl]-naphthalen-2-ol.

64. The formulation of claim 59, wherein the isoquinoline is 1-(4′-pyrrolidino-ethoxyphenyl)-2-(4″-fluoroophenyl)-6-hydroxy-1,2,3,4-tetrahydroisoquinoline, or 1-(4′-pyrrolidino-ethoxyphenyl)-2-phenyl-6-hydroxy-1,2,3,4 tetrahydroisoquinoline.

65. The formulation of claim 59, wherein the benzopyran is selected from the group consisting of 4-fluoroalkyl-2H-benzopyrans and 2,3 diaryl-2H-1-benzopyrans.

66. The formulation of claim 59, wherein the steroid is selected from the group consisting of tibolone, diethylstilbestrol, moxestrol, N-butyl-3,17-dihydroxy-N-methyl-estra-1,3,5(10)-triene-7-undecanamide, fulvestrant, 19-nor-progesterones, and 19-nor-testosterones.

67. The formulation of claim 59, wherein the coumarin is selected from the group consisting of 3-phenyl-4-[4-(2-(piperadin-1-yl))ethoxy]-benzyl-7-hydroxycoumarin, 3-(4-chlorophenyl)-4-[4-(2-(piperadin-1-yl))ethoxy]-phenyl-7-hydroxycoumarin, di ethyl amino substituted coumarin, chroman, centchroman, and levormeloxifene.

68. The formulation of claim 59, wherein the phytoestrogen is genistein.

69. The formulation of claim 59, wherein the diphenol is hexestrol.

70. A biodegradable adhesive film for application to the vulvar region of a female patient suffering from dyspareunia, comprising a therapeutically effective amount of nitroglycerin in a biodegradable adhesive polymer composition effective to provide for sustained drug release.

71. The film of claim 70, wherein the polymer composition is comprised of a polymer selected from the group consisting of polyurethanes, poly(lactic acid), poly(glycolic acid), a poly(ortho esters), polyanhydrides, polyphosphazenes, and mixtures and copolymers thereof.

72. The film of claim 70, wherein the polymer composition is comprised of a polymer selected from the group consisting of polyurethanes containing peptide linkages.

73. The film of claim 70, wherein the polymer composition is comprised of a polymer selected from the group consisting of polyurethane block copolymers containing peptide linkages.

74. The film of claim 70, wherein the polymer composition is comprised of a mixture of a polyurethane and a polylactide.

75. The film of claim 70, wherein the polymer composition is comprised of a copolymer of an acrylate monomer and a mono- or di-saccharide.

76. A packaged kit for a patient to use in the treatment of dyspareunia, comprising: a pharmaceutical formulation containing a therapeutically effective amount of nitroglycerin; a container housing the pharmaceutical formulation during storage and prior to administration; and instructions for carrying out drug administration in a manner effective to treat dyspareunia.

77. The packaged kit of claim 76, wherein the pharmaceutical formulation is a rapid-release dosage form containing a unit dosage of nitroglycerin, the unit dosage being a therapeutically effective dosage for treatment of dyspareunia.
